(19) United States
(12) Patent Application Publication
(10) Pub. No.: US 2021/0244348 A1
(43) Pub. Date: Aug. 12, 2021

(54) SKIN RASH METER

(71) Applicant: Archibald James DeMone, Westford, MA (US)

(72) Inventor: Archibald James DeMone, Westford, MA (US)

(21) Appl. No.: 16/784,040

(22) Filed: Feb. 6, 2020

(51) Int. Cl. A61B 5/00

(52) U.S. Cl. CPC A61B 5/445; A61B 5/443; A61B 2562/08; A61B 5/742; A61B 2560/0214; A61B 5/0064

(57) ABSTRACT

The disclosed skin rash meter quantitatively measures the intensity of skin rash, the redness of skin due to the pooling of blood near the surface of skin or a jaundice condition of the skin. This is necessary to determine if the condition is getting better or worst due to the patient's condition or the patient's response to medication being used for treatment of the condition. Presently, this measurement is taken as a subjective observation by medical staff which is very unreliable. This invention measures the severity of these types of conditions and presents a number that is a repeatable scientific measurement of the severity of the condition. This measurement can be kept as a history of the severity of the condition from which a trend can be determined and decisions can be made of the correct treatment and prognosis of the condition.

### BACKGROUND OF THE INVENTION

A need exists in medicine to quantitatively measure the intensity of skin rash, the redness of skin due to the pooling of blood near the surface of skin and a jaundice condition of the skin. This is necessary to determine if the condition is getting better or worst due to the patient's condition or the patient's response to medication being used for treatment of the condition. Presently, this measurement is taken as a subjective observation by medical staff which is very unreliable.

### BRIEF SUMMARY OF THE INVENTION

This invention measures the severity of these types of conditions and presents a number that is a scientific measurement of the severity of the condition.

This measurement can be kept as a history of the severity of the condition from which a trend can be determined and decisions can be made of the correct treatment and prognosis of the condition.

This invention operates typically in the following manner. A blue wavelength light is projected onto the skin in the area of interest from a blue light source such as a blue Light Emitting Diode (LED), a blue wavelength LASER or a white light source filtered with a blue light pass filter. The amount of blue light reflected off of the skin is measured with a photodetector and the mount of blue light absorbed and reflected by the skin is a measure of the redness of the skin. The redder the skin, the more blue light it absorbs and the less blue light is reflected and sent back to the photodetector. The reflected light can be filtered with a blue light pass filter to help eliminate non blue light or the photodetector can be made of silicon which favors blue light. This device is calibrated by measuring human skin types with varying degrees of rash, redness or jaundice and it presents that measurement as a number to the operator. This results in repeatable and quantitatively measures these skin conditions rather than relying upon subjective observation by a human and often different humans are used to obtain a history or trend of the condition.

This measuring device can be connected to computer through a wire or wireless connection. It can also incorporate a bar code reader to facilitate connecting the patient ID to the skin measurement record.

Different skin type calibrations can be incorporated in the device and can be selected by the operator before a measurement is taken.

Skin rash and jaundice measurements can be taken by pressing the device against the skin but some skin redness measurements must be taken with the device in a standoff noncontact position. This standoff arrangement can be obtained by a set of legs that contact the skin away from the area to be measured with a room light blocking skirt surrounding the area of interest.

Single use sheaths are needed to shield the device from the skin so as not to transfer bacteria from one patient to another patient. These would be of two configurations. The first configuration would be for rash measurements and are pressed directly against the skin. The second configuration has standoff legs so as not to put pressure on the skin near the area to be measured. It also has a soft skirt surrounding the area of interest to prevent room light from affecting the measurement.

### BRIEF DESCRIPTIONS OF THE DRAWINGS

For a fuller understanding of the nature and objects of the present invention, reference should be had to the following detailed description taken in connection with the accompanying drawings in which the same reference numerals are used to indicate the same or similar parts wherein:

FIG. 1 shows a sectional view of one embodiment of a meter for measuring skin rash redness or jaundice constructed according to the invention;

FIG. 2 is a flow diagram of the light paths and signal paths of the invention;

FIG. 3 is a view of the display and control panel of this embodiment of the invention;

FIG. 4 is a view showing the light emitting and light detection apparatus of this embodiment of the invention;

FIG. 5 is a view of a single use protective sheath attached to the invention when the invention is pressed directly to the skin so as not to transfer bacteria from one patient to another patient.

FIG. 6 is a view of a single use protective sheath attached to the invention when the invention is held away from being pressed directly to the skin so as not to transfer bacteria from one patient to another patient.

### DETAILED DESCRIPTION OF THE DRAWINGS

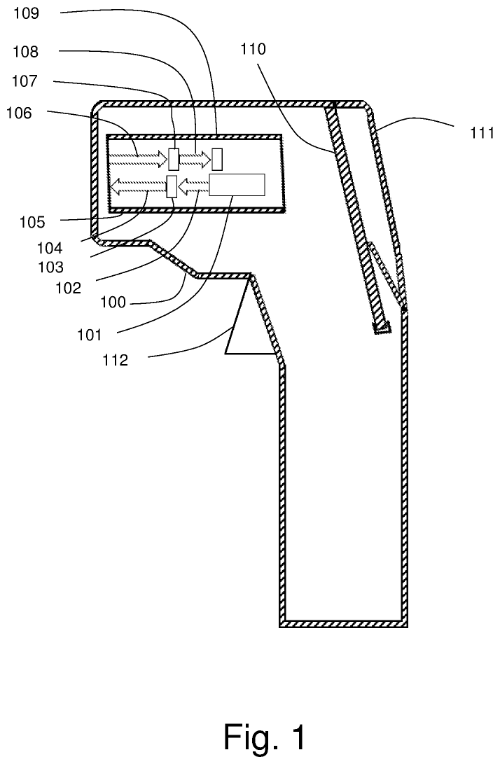

FIG. 1 shows a sectional view of a skin rash, skin redness or jaundice meter assembly 100 constructed according to at least one aspect of the present invention. As will be discussed in greater detail below, meter assembly 100 provides a repeatable determinate measurement of skin rash, skin redness or jaundice.

Meter assembly 100 includes a light source 101, an optional light pass filter 103, a light baffle 105, an optional light pass filter 107, a photo detector 109, an electronics assembly 110, a numeric display and control panel 111 and an on/off switch 112.

In operation, light source 101 generates the color of light required to produce the most absorption and least reflection of the light from the skin condition that is being measured. 102 and 104 indicate the light path directed toward the skin and 106 and 108 indicate the light path reflected from the skin and returned to the photodetector 109. Optional light pass filters 103 and 107 further select the color of light required. Light baffle 105 blocks room light that will cause measurement error. Photo detector 109 produces an electronic signal that is proportional to the light impinging upon it. Electronics 110 quantifies the electronic signal output of photo detector 109 and provides the electronic signals to the display 111. Display and control panel 111 creates a numeric display representing the measurement of the skin condition being measured, symbols indications the modes of the device and controls such as push buttons to set the device in the desired modes.

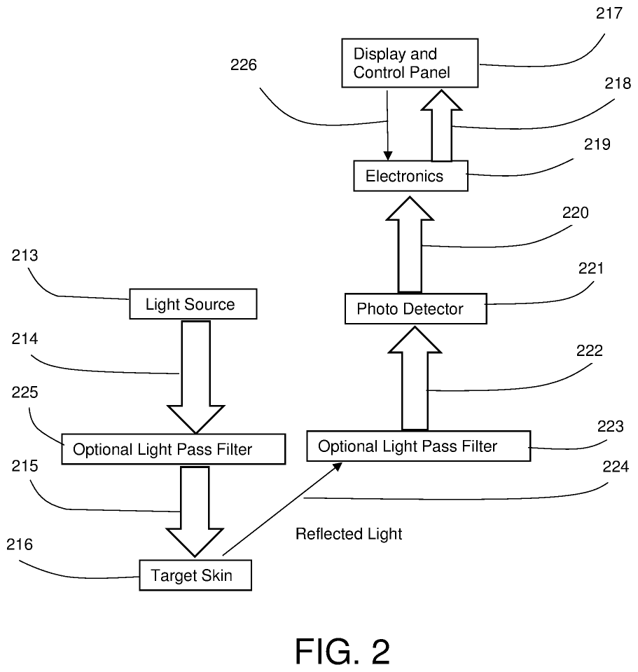

FIG. 2 shows a flow diagram of the skin rash, skin redness or jaundice meter constructed according to at least one aspect of the present invention.

Light source 213 emits light containing the colors used in the measurement of interest. Optional light pass filters 214 and 223 select the light color of interest. The light path traveling towards the skin are indicated by 214 and 215 and the light paths of the light reflected from the target skin 216 are shown as 220, 222 and 224. Photo detector 222 converts the reflected light impinging upon it into an electrical signal which is sent to the electronics 219. Electronics 219 converts the electrical signal from photo detector 222 into a number which is displayed on display and control panel 217 for viewing by the operator. Display and control panel 217 also contains input devices such as push buttons that feed electronic signals 226 to electronics 219 to set the internal calibration curves being used depending on the skin type and the type of measurement being taken.

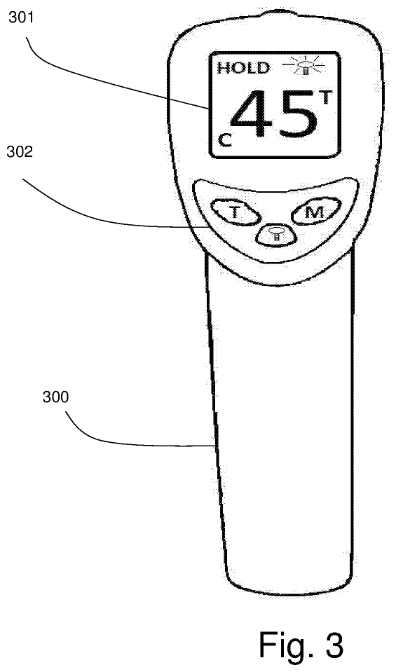

FIG. 3 shows a display side view of a skin rash, skin redness or jaundice meter assembly 300 constructed according to at least one aspect of the present invention.

Meter assembly 300 includes a display 301 which displays a number indicating the level of the skin condition being measured, the type of skin calibration curve being used, the operating mode being used, the display mode and the display lighting mode.

Control panel 302 contains push buttons which are used to select the skin type calibration curve to use, the operating mode, contact mode or standoff mode and the lighting mode for the display, room light or back lighting.

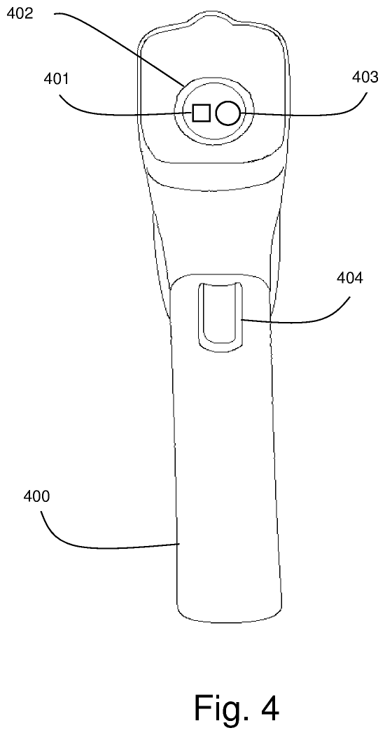

FIG.4 shows a light transmitting and receiving side view of a skin rash, skin redness or jaundice meter assembly 400 constructed according to at least one aspect of the present invention.

In operation, light source 403 generates the color of light required to produce the most absorption and least reflection of the light from the skin condition that is being measured. Photo detector 401 converts the reflected light impinging upon it into an electrical signal which is sent to the electronics. Light baffle 402 blocks room light that will cause measurement error. On/off switch 404 provides power to the unit during measurements and during the displaying of results.

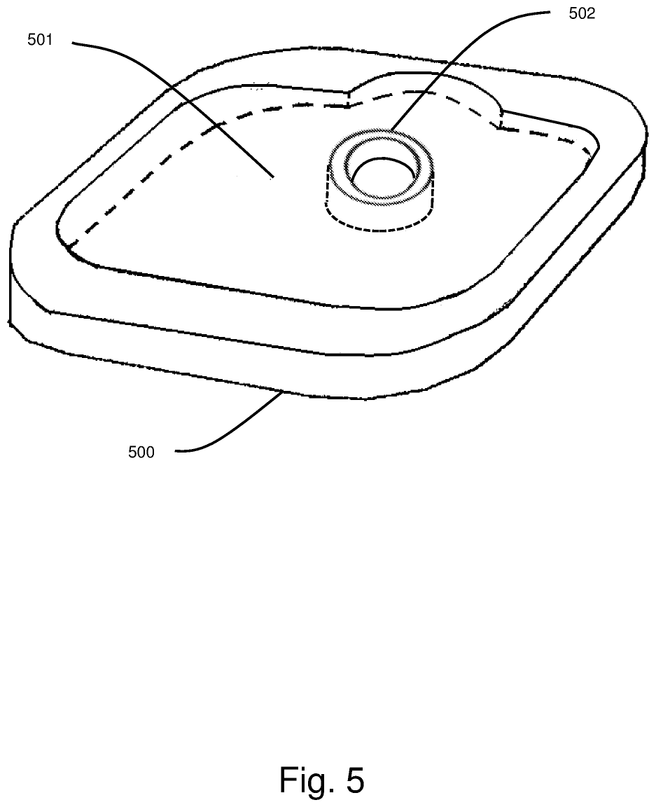

FIG. 5 is a view of a single use protective sheath 500 which is attached to the invention when the invention is pressed directly to the skin so as not to transfer bacteria from one patient to another patient. Membrane 501 separates the inventions outer surface from the skin. Light baffle 502 blocks room light from impinging on the photo detector and causing error in the measurement.

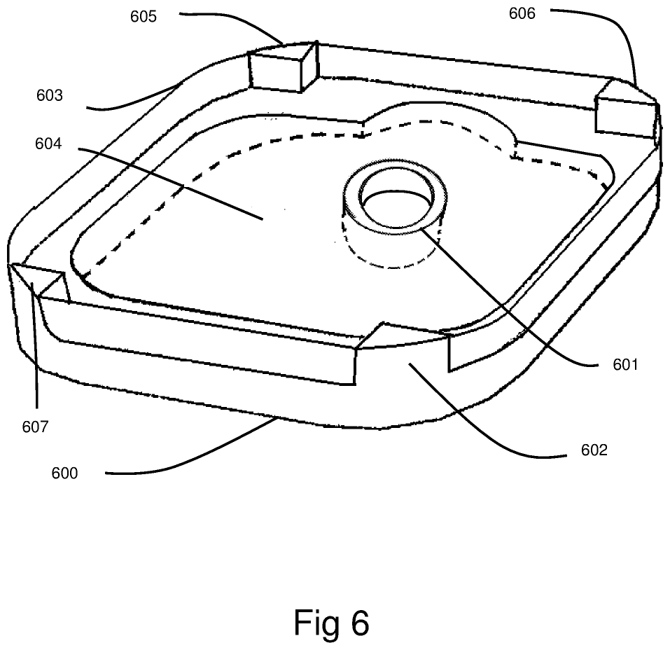

FIG. 6 is a view of a single use protective sheath 600 which is attached to the invention when the invention is held away from being pressed directly to the skin so as not to transfer bacteria from one patient to another patient. Standoffs 602, 605, 606 and 607 hold the invention away from the skin so as not to drive blood away from the area being measured when the medical condition is a result of the pooling of blood in the skin. Light baffle 601 blocks room light from impinging on the photo detector and causing error in the measurement. Membrane 604 separates the inventions outer surface from the skin. Flexible skirt 603 blocks room light from impinging on the photo detector and causing error in the measurement.

Since certain changes may be made in the above apparatus without departing from the scope of the invention herein involved, it is intended that all matter contained in the above description or shown in the accompanying drawings shall be interpreted in an illustrative and not a limiting sense.

What is claimed is:

1. A skin rash, skin redness and skin jaundice meter assembly comprising
(A) An external enclosure defining an interior cavity
(B) A light source
(C) A photonic detector
(D) An electronic processing unit
(E) A numeric and symbol display
(F) A control panel
(G) A power switch
(H) A room light baffle
(I) A power source.

2. A skin rash, skin redness and skin jaundice meter assembly according to claim 1, further comprising a light pass filter through which the light from the light source passes to enhance the color of light that enhances the accuracy and repeatability of the invention.

3. A skin rash, skin redness and skin jaundice meter assembly according to claim 1, further comprising a light pass filter through which the light reflected from the skin passes to enhance the color of light that enhances the accuracy and repeatability of the invention.

4. A skin rash, skin redness and skin jaundice meter assembly according to claim 1, further comprising a battery as a power source.

5. A skin rash, skin redness and skin jaundice meter assembly according to claim 1, further comprising an electronic internal or external power supply powered by the power grid.

6. A skin rash, skin redness and skin jaundice meter assembly according to claim 1, further comprising a data communications connection to a computer using a wired connection.

7. A skin rash, skin redness and skin jaundice meter assembly according to claim 1, further comprising a data communications connection to a computer using a wireless connection.

8. A skin rash, skin redness and skin jaundice meter assembly according to claim 1, further comprising an electronic controller capable of calculating and displaying on the display numeric values representing correct values of the measurement for different skin types.

9. A skin rash, skin redness and skin jaundice meter assembly according to claim 1, further comprising a bar code reader.

10. A single use protective sheath attached to the invention when the invention is pressed directly to the skin so as not to transfer bacteria from one patient to another patient.

11. A single use protective sheath which is attached to the invention when the invention is held away from being pressed directly to the skin so as not to transfer bacteria from one patient to another patient.
